(19) United States
(12) Patent Application Publication
(10) Pub. No.: US 2022/0213506 A1
(43) Pub. Date: Jul. 7, 2022

(54) EXPRESSION OF ANTIGEN-BINDING PROTEINS IN THE NERVOUS SYSTEM

(71) Applicant: SANOFI, Paris (FR)

(72) Inventors: Bradford ELMER, Arlington, MA (US); Zhi-Yong YANG, Newton, MA (US); Gary NABEL, Chestnut Hill, MA (US)

(73) Assignee: SANOFI, Paris (FR)

(21) Appl. No.: 17/611,849

(22) PCT Filed: May 15, 2020
(86) PCT No.: PCT/IB2020/054642

§ 371 Date: Nov. 16, 2021

Related U.S. Application Data
(60) Provisional application No. 62/848,659, filed on May 16, 2019.

(30) Foreign Application Priority Data
Oct. 8, 2019 (EP) 19306310.4

(51) Int. Cl. C12N 15/86; C07K 14/47; C07K 16/18; C12N 5/0793; C12N 5/079

(52) U.S. Cl. CPC C12N 15/86; C07K 14/4711; C07K 16/18; C12N 2750/14143; C12N 5/0622; C07K 2317/24; C07K 2317/622; C12N 5/0619

(57) ABSTRACT

Provided herein are recombinant vectors that express bivalent binding members and methods of using the vectors to modify cells of the nervous system to express the binding members in the brain of patients having a neurological disease such as a neurodegenerative disease.

### SEQUENCE LISTING

The instant application contains a Sequence Listing which has been submitted electronically in ASCII format and is hereby incorporated by reference in its entirety. Said ASCII copy, created on Apr. 30, 2020, is named 022548_WO060_SL.txt and is 7,612 bytes in size.

### BACKGROUND OF THE INVENTION

Alzheimer's disease (AD) is characterized by progressive neurodegeneration leading to memory loss and a decline in cognitive function. Its pathological features include the accumulation of extracellular amyloid plaques and intraneuronal tau fibrils. Therapies targeting amyloid beta (Aβ) have been under active investigation for many years due to its genetic and pathologic involvement in AD (Tcw and Goate, Cold Spring Harb Perspect Med. (2017) 7(6): pii a024539). While increased levels of amyloid precursor protein (APP) and Aβ are associated with AD pathogenesis, Aβ peptides exist in different conformations and fibrillary status, and it is unclear which species should be targeted for therapeutic benefit (Benilova et al., Nat Neurosci. (2012) 15:349-57).

Despite this uncertainty, passive immunotherapy against different forms of Aβ has been extensively tested in the clinic; however, these approaches have been hampered by additional problems. First, the blood brain barrier (BBB) restricts transport of large biomolecules, necessitating the injection of high doses in the periphery to reach therapeutically relevant levels in the brain. At high doses, several anti-Aβ antibodies in clinical trials caused adverse reactions typified by amyloid-related imaging abnormalities (ARIA); these adverse reactions are thought to be caused by antibody accumulation at sites of vascular amyloid, triggering local inflammation via Fc-dependent effector functions (Mo et al., Ann Clin Transl Neu. (2017) 4:931-42). Second, there is the need to maintain levels above a minimal therapeutic dose, requiring long-term passive immunotherapy that requires patient engagement and compliance, as well as a significant cost of goods.

Gene transfer into the central nervous system (CNS) allows for production of therapeutic protein within neuronal cells and therefore circumvents the BBB. AAV-mediated expression of either whole immunoglobulins (IgG) or single chain variable fragments (scFv) has been attempted within the CNS, but both of these approaches have inherent limitations (Sudol et al., Mol Ther. (2009) 17:2031-40; Ryan et al., Mol Ther. (2010) 18:1471-81; Levites et al., J Neurosci. (2006) 26:11923-28; Levites et al., J Neurosci. (2015) 35:6265-76; Kou et al., JAD. (2011) 27:23-38; Fukuchi et al., Neurobio Dis. (2006) 23:502-11; Liu et al., J Neurosci. (2016) 36:12425-35). Heavy and light chain expression of IgG in the CNS has only been accomplished using a self-cleavable F2A sequence to generate both chains from a single-promoter cassette. The F2A peptide remains attached to either heavy or light chain and is potentially immunogenic (Saunders et al., J Vir. (2015) 89:8334-45). Gene-based delivery of scFv proteins, on the other hand, is often accompanied by a substantial loss in affinity due to the loss of valency. Removal of the Fc region also results in a loss of FcRn binding, causing shorter half-life in the periphery and reduced efflux of antigen (Ag)-bound scFvs from the brain via reverse transcytosis (Deane et al., J Neurosci. (2005) 25:11495-503; Boado, et al., Bioconjug Chem. (2007) 18:447-55; Zhang et al., J Neuroimm. (2001) 114:168-72; Schlachetzki et al., J Neurochem. (2002) 81:203-6). Hence, antibody therapies for CNS diseases such as Alzheimer's disease hold promise but have been limited by the problems of introducing the therapeutic proteins to the diseased brain. Accordingly, there exists a need for improving central nervous system access for antibody-based therapies.

### SUMMARY OF THE INVENTION

The present disclosure provides a method of expressing a bivalent binding member in a cell of the nervous system, comprising introducing into the cell an expression cassette encoding a polypeptide comprising an antibody heavy chain variable domain (V_{H}), an antibody light chain variable domain (V_{L}), and an IgG Fc region, wherein the V_{H} and the V_{L} form an antigen-binding site that binds specifically to a target protein, and upon expression in the cell, two molecules of the polypeptide form a disulfide-bonded homodimeric bivalent binding member specific for the target protein.

In some embodiments, the cell of the nervous system is a neuron, a glial cell, an ependymal cell, or a brain epithelial cell. In further embodiments, the glial cell is selected from an oligodendrocyte, an astrocyte, a pericyte, a Schwann cell, and a microglia cell. In some embodiments, the cell is a human cell, such as a cell in the brain of a human patient.

In some embodiments, the target protein is a protein expressed in the brain and may be amyloid beta peptide (Aβ), tau, SOD-1, TDP-43, ApoE, or α-synuclein.

In some embodiments, the polypeptide comprises, from N-terminus to C-terminus, (i) the V_{H}, a peptide linker, and the V_{L}; or the V_{L}, a peptide linker, and the V_{H}; and (ii) the IgG Fc region. In further embodiments, the peptide linker comprises the sequence GGGGS (SEQ ID NO: 3); for example, the peptide linker has the sequence of [G₄S]₃ (SEQ ID NO: 2).

In some embodiments, the bivalent binding member of the present disclosure binds to neonatal Fc receptor (FcRn), but it does not bind to an Fc gamma receptor due to one or more mutations in the IgG Fc region.

In some embodiments, the present method comprises administering a viral vector containing the expression cassette. The viral vector may be is a recombinant virus. In further embodiments, the recombinant virus is introduced to the brain of a patient via intracranial injection, intrathecal injection, or intracisterna-magna injection. The recombinant virus may be, for example, a recombinant adeno-associated virus (rAAV), e.g., rAAV of serotype 1 or 2.

In some embodiments, expression of the polypeptide is under the transcriptional control of a constitutively active promoter or an inducible promoter.

The present methods may be used to treat a patient with a neurodegenerative disease, e.g., Alzheimer's disease, cerebral amyloid angiopathy, synucleopathy, tauopathy, or amyotrophic lateral sclerosis (ALS).

In another aspect, the present disclosure provides a method of treating a neurodegenerative disease, comprising administering to a patient in need thereof a therapeutically effective amount of a composition comprising the viral vector disclosed herein that expresses a bivalent binding member of the present disclosure.

In another aspect, the present disclosure provides a bivalent binding member for use in treating a patient in need thereof, and a use of a bivalent binding member for the manufacture of a medicament for the treatment of a patient in need thereof, wherein the patient has, for example, a neurodegenerative disease such as Alzheimer's disease, cerebral amyloid angiopathy, synucleopathy, tauopathy, or ALS.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A-C show the construction and characterization of an AAV-IgG vector.

FIG. 1A shows the vector design for full heavy and light chain expression. The size of the genome is indicated.

FIG. 1B, left panel, shows durable expression and secretion of AAV-αAβ IgG from the brain as compared to huIgG measured from PBS injected control mice. Graphed points represent the mean +/−SEM, n=8 mice per group. The right panel shows the dynamics of AAV-mediated expression of AAV-αAβ IgG in the brain versus traditional peripherally administered αAβ IgG. Graphs show the mean +/−SEM. **p<0.01, 1-way ANOVA at 7 weeks post injection, n=5 mice per time point.

FIG. 1C shows a colored micrograph of neurons expressing the huIgG transgene throughout the hippocampus (CA2 shown in detail), with some GFAP+ astrocytes nearby also expressing huIgG. Cc=corpus callosum. Green: human IgG (huIgG). Red: glial fibrillary acidic protein (GFAP). Blue: DAPI.

FIGS. 2A and B show antigen binding by AAV-αAβ IgG in a mouse model of Alzheimer's disease.

FIG. 2A Shows the study design for intracranial (AAV-αAβ IgG or AAV-IgG Control) and peripheral dosing (αAβ IgG).

FIG. 2B shows the expression of AAV-αAβ IgG or AAV-IgG Control throughout the hippocampus and overlying cortex. Images on the right panel show IgG binding to plaques in frontal cortex. Scale bars=10 μm. Blue: DAPI. Green: huIgG. Red: 4G8+GFAP.

FIGS. 3A-C show the evaluation of AAV-αAβ IgG neuronal expression and neurotoxicity.

FIG. 3A, left panel shows the detected peptides from huIgG heavy and light chain from hemibrain lysates of SCID mice injected with AAV-αAβ IgG compared to animals injected with PBS (Sham), or Sham brain homogenate spiked with equivalent levels of huIgG as in the AAV-αAβ IgG group. The right panel shows the quantification of functional huIgG compared to total huIgG expressed in SCID mice either centrally or peripherally. Data are presented as mean +/−SEM. **p<0.01, unpaired Student's t-test.

FIG. 3B, left panel shows H&E staining of C57BL/6 mouse brain hippocampus following intra-hippocampal AAV-αAβ msIgG expression for 16 weeks compared to PBS control. Inset shows detail, arrows point to representative hyaline inclusions. Scale bar=100 μm. Results are summarized in the right panel table as the number of animals scored with or without this pathology.

FIG. 3C shows evidence of neuroinflammation by immunohistochemistry (IHC) Glial fibrillary acidic protein (GF AP) analysis relative to PBS. The left panel shows quantitative (IHC) for GFAP+ area. On the right panel, each circle represents one mouse. Bars indicate group mean +/−SEM of GFAP+ area normalized to PBS. ***p<0.001, unpaired Student's t-test, n=8 mice per group.

FIGS. 4A-C show the construction and characterization of an AAV-scFv-IgG vector.

FIG. 4A, left panel shows a schematic of the scFv-IgG design. The middle panel shows that reducing or non-reducing SDS-PAGE analysis of the purified scFv-IgG demonstrated purity and proper disulfide-dependent dimerization of the protein. The right panel table compares antigen binding affinity (M) of the scFv-IgG versus the IgG format.

FIG. 4B, left panel shows serum expression of the AAV-scFv-IgG as measured by antigen enzyme-linked immunosorbent assay (ELISA) one month following peripheral IV injections of AAV into C57BL/6 mice. The right panel shows brain expression of the AAV-scFv-IgG. ***p<0.001, unpaired Student's t-test, n=5 mice per group for intracranial injection, 2 mice per group for IV injection.

FIG. 4C, left panel shows hippocampal targeting of the vector, and transduction throughout the hippocampal formation following IHC on sagittal sections of mouse brain taken from the same animals as in FIG. 4B, right panel. The right panel shows ELISA-based quantification of scFv-IgG in different dissected brain regions after bilateral hippocampal injection of AAV-scFv-IgG. Hipp=hippocampus. Ctx=overlying cortical regions. Str=striatum.

FIGS. 5A-C show the expression, diffusion, and plaque binding of the anti-Aβ scFv-IgG.

FIG. 5A shows a whole scan of hippocampus and overlying cortex of adult mice one-month post-injection with anti-Aβ AAV-scFv-IgG. Sections were immunostained for AP plaques (4G8, red) and 6xHis (SEQ ID NO: 9) (green). Scale bar=300 μm. Cc=corpus callosum. Images on the right panel show individual plaque ROIs (numbered in A) proximal (1) to distal (6) from the site of injection. Abundant plaque formation was observed throughout the cortex (left panel) and staining with an anti-His antibody co-localized with plaques (right panel). Regions of interest (ROIs) are 150 μm in diameter. Red: 4G8. Green: anti-HIS antibody. Blue: DAPI.

FIG. 5B, left panel shows an outline of the study design. Images on the right panel show the hippocampus from coronal sections of AAV-injected mice. IHC revealed labeling throughout the hippocampus on the injected side (red arrow), with additional transduction of the contralateral hippocampus. AAV-empty injected brain did not show any anti-His labeling. Scale bar=1 mm.

FIG. 5C shows the quantification of plaque deposition in cortex and hippocampus of animals from each respective group. N=10-13 animals per group, 3 sections per animal. ***p<0.001 one-way ANOVA with multiple comparisons. Errors bars represent the standard error of the mean (SEM).

### DETAILED DESCRIPTION OF THE INVENTION

The present disclose provides a method of expressing a bivalent binding member in a cell of the nervous system without the side effects seen with current expression methods. Cells of the neural system do not naturally express antibodies. Prior studies have shown that expression of full antibodies in the brain causes neurotoxicity. Compared to conventional methods of expressing wildtype IgG in brain cells, the expression methods of the present disclosure afford unexpectedly higher yield (e.g., two times or more higher) and lower toxicity (e.g., as indicated by the lack of detectable intraneuronal hyaline protein accumulation at the injected site). Without being bound by theory, the inventors contemplate that cells in the nervous system are not equipped to express and assemble native antibody efficiently, and that unpaired antibody chains form inclusion bodies that are toxic to cells; the present expression methods, however, overcome this problem by reducing the number of the polypeptide chains to be expressed from two to one. The present expression methods also are advantageous over prior methods of expressing scFvs in the brain, because the present methods allow the expression of a binding molecule that has higher avidity and better pharmacokinetic profiles (e.g., half-life).

### Cells of the Nervous System

The present disclosure provides a method of a cell of the nervous system to express (e.g., including secretion) a bivalent molecule that is specific to a target protein expressed in the nervous system, such as the central nervous system including the brain and the spinal cord. Cells of the nervous system for expressing a binding member of the present disclosure may be of any cell type in the nervous system, such as any cell type in the brain. For example, the present method may express the binding member in a neuronal cell (e.g., an interneuron, a motor neuron, a sensory neuron, a brain neuron, a dopaminergic neuron, a cholinergic neuron, a glutamatergic neuron, a GABAergic neuron, or a serotonergic neuron); a glial cell (e.g., an oligodendrocyte, an astrocyte, a pericyte, a Schwann cell, or a microglia cell); an ependymal cell; or a brain epithelial cell. In some embodiments, these cells are human cells. The cells may also be those located in any targeted region of the human brain, such as the hippocampus, the cortex, the basal ganglia, the midbrain, or the hindbrain.

### Bivalent Binding Members

The present disclosure provides a bivalent binding member that is expressed in a cell of the nervous system and binds a target antigen expressed in the nervous system such as the brain. The target antigen may be, for example, a protein that mediates a neurological disease such as a neurodegenerative disease. Antigens of interest include, without limitation, amyloid beta peptide (Aβ), tau, SOD-1, TDP-43, ApoE, and α-synuclein.

The bivalent binding member is a homodimer of a polypeptide chain, where the polypeptide chain comprises an antigen-binding domain and a constant region of an antibody (e.g., a hinge region, a CH2 domain, and a CH3 domain of an IgG such as a human IgG). The homodimer thus comprises two antigen-binding sites and an Fc domain of an antibody.

In some embodiments, the antigen-binding domain of the polypeptide chain is a single-chain Fv (scFv) domain. The scFv domains comprises an antibody heavy chain variable region (VH) and an antibody light chain variable region (VL), where the VH and the VL are optionally separated by a peptide linker and interact to form an antigen-binding site. Methods of obtaining an scFv polypeptide to an antigen of interest are well known in the art. For example, one can screen a phage display library to obtain VH and VL combinations that bind to the antigen with high affinity, or one can derive the VH and VL sequences from a preexisiting antibody that specifically binds to the antigen.

The antigen-binding domain, such as an scFv domain, can be fused, with or without a peptide linker (e.g., such as those exemplied herein, including a 9-Gly repeat linker (SEQ ID NO: 7)), to a constant region of an antibody, where the constant regions of the two polypeptide chains form an antibody Fc domain through one or more disulphide bonds. As used herein, the term “Fc region” or “Fc domain” refers to a portion of a native immunoglobulin formed by the dimeric association of the one or more constant domains of the immunoglobulin.

In some embodiments, each polypeptide sequence of the Fc domain may include the portion of a single immunoglobulin (Ig) heavy chain beginning in the hinge region just upstream of the papain cleavage site and ending at the C-terminus of the Ig heavy chain. The Fc domain may comprise a hinge region, the CH2 and CH3 of an immunoglobulin. Depending on the Ig isotype from which the Fc domain is derived, the Fc domain may include additional constant domains (e.g, a CH4 domain of IgE or IgM). The Fc domain may contain mutations relative to wildtype sequences to, e.g., enhance the fusion dimeric protein's stability (e.g., half-life) and/or to modify the fusion mideric proteins' effector functions. The mutations may be additions, deletions, or substitutions of one or more amino acids.

In some embodiments, the Fc domain is derived from an IgG such as a hman IgG, and may be of any IgG subtype, such as of human IgG1, IgG2, IgG3, or IgG4 subtype. In such cases, the scFv-Fc of the present disclosure is also termed scFv-IgG. The Fc domain may comprise the entire hinge region or only a part thereof of an IgG, e.g., an IgG1, IgG2, IgG3, or IgG4 hinge region. In some embodiments, the Fc domain is derived from a human IgG1 and comprises mutations L234A and L235A (“LALA”) (EU numbering) such that the Fc domain does not bind to high affinity Fc gamma (γ) receptor(s) and has reduced ADCC/CDC effector functions. Other Fc mutations that may be introduced to human IgG1 include, without limitaiton, N297Q, N297A, N297G, C220S/C226S/C229S/P238S, C226S/C229S/E233P/L234V/L235A, and L234F/L235E/P331S (EU numbering). See, e.g., Wang et al., Protein Cell. (2018) 9(1):63-73; Strohl, Curr Opin Biotechnol. (2009) 20(6):685-91; Johnson et al., Nat Med. (2009) 15(8):901-6. In some embodiments, the binding member has a hinge region from human IgG4, wherein the hinge region contains an S228P mutation (EU numbering) to reduce dissocation of two polypeptide chains of the binding member. In certain embodiments, the Fc domain is derived from a human IgG4 and comprises mutations S228P and L235E (EU numbering; corresponding to S241P and L248E in Kabat numbering), which reduce Fcγ half-molecule exchange and effector function, respectively (Reddy et al., J Imm. (2000) 164:1925-33). Loss or reduction of ADCC/CDC effector functions allows the binding member to bind to the target antigen without causing cytotoxicity or eliciting unwanted inflammation in the nervous sytem. In further embodiments, the modified Fc domain retains its ability to bind to FcRn, a neonatal Fc receptor. Retension of the FcRn binding ability allows an antigen-bound binding member to be removed from the nervous system such as the brain by FcRn-mediated reverse transcytosis.

In some embodiments, the VH and VL domains of the scFv-Fc binding member, and/or the scFv and Fc domains of the binding member, are linked via a peptide linker. Suitable peptide linkers are well known in the art. See, e.g., Bird et al., Science (1988) 242:423-26; and Huston et al., PNAS. (1988) 85:5879-83. The peptide linker may be rich in glycine and/or serine. Examples of peptide linkers are G, GG, G3S (SEQ ID NO: 1), G45 (SEQ ID NO: 3), and [G₄S]n (n=1, 2, 3, or 4; SEQ ID NO: 4). In some embodiments, a 9-Gly repeat linker (SEQ ID NO: 7) is used to link an scFv to an IgG portion in an scFv-IgG format of the present disclosure.

In particular embodiments, an scFv-IgG of the present disclosure is designed to have the variable domains linked via a peptide linker using a [G₄S]₃-type peptide linker (SEQ ID NO: 2). [G₄S]₃-type linkers (SEQ ID NO: 2) have been widely used to link variable domains in an scFv structure (Huston, supra). As used herein, a [G₄S]₃-type linker (SEQ ID NO: 2) refers to [G₄S]₃ (SEQ ID NO: 2) or a functional variant thereof (e.g., a peptide linker having up to four amino acid modifications (e.g., insertions, deletions, and/or substitutions) from [G₄S]₃ (SEQ ID NO: 2)). By way of examples, a functional variant of [G₄S]₃ (SEQ ID NO: 2) may be the amino acid sequence SGGGSGGGGSGGGGS (SEQ ID NO: 5) or the amino acid sequence GGGGSGGGGXGGGGYGGGGS (X=S, A or N, and Y=A or N; SEQ ID NO: 6).

In some embodiments, the amino acid sequence of the linkers may be modified. Modifications can include deletions or insertions that change the linker length (e.g., to adjust for flexibility), or amino acid substitutions, including, for example, from Gly to Ser or vice versa.

A scFv-Fc polypeptide against Aβ is shown below, merely to illustrate one format of the scFv-Fc polypeptide. The following sequence, from N-terminus to C-terminus, contains a signal peptide (italicized), VL, [G₄S]₃ linker (SEQ ID NO: 2) (underlined), VH, G₉ (SEQ ID NO: 7) (boxed), IgG1 hinge and Fc domain, and a short linker attached to a 6xHis tag (SEQ ID NO: 9) (boldface).

(SEQ ID NO: 8)
MDSKGSSQKG SRLLLLLVVS NLLLPQGVLA SEIVMTQTPL SLPVSLGDRA
SISCRSGQSL VHSNGNTYLH WYLQKPGQSP KLLIYTVSNR FSGVPDRFSG
SGSGSDFTLT ISRVEAEDLG VYFCSQNTFV PWTFGGGTKL EIKRTSSGGG
GSGGGGSGGG GSEVQLQQSG PEVVKPGVSV KISCKGSGYT FTDYAMHWVK
QSPGKSLEWI GVISTKYGKT NYNPSFQGQA TMTVDKSSST AYMELASLKA

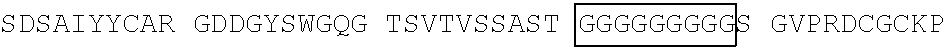

CICTVPEVSS VFIFPPKPKD VLTITLTPKV TCVVVDISKD DPEVQFSWFV
DDVEVHTAQT QPREEQFAST FRSVSELPIM HQDWLNGKEF KCRVNSAAFP
APIEKTISKT KGRPKAPQVY TIPPPKEQMA KDKVSLTCMI TDFFPEDITV
EWQWNGQPAE NYKNTQPIMD TDGSYFVYSK LNVQKSNWEA GNTFTCSVLH
EGLHNHHTEK SLSHSPGSGS GSGSHHHHHH

### Expression of Binding Members in the Nervous System

An expression construct containing an expression cassette for the binding member may be introduced to the cells of the nervous system by well-known methods. For example, for in vivo or ex vivo delivery, a viral vector may be used. In some embodiments, the expression vector remains present in the cell as a stable episome. In other embodiments, the expression vector is integrated into the genome of the cell. The expression vectors may include expression control sequences such as promoters, enhancers, transcription signal sequences, and transcription termination sequences that allow expression of the coding sequence for the binding member in the cells of the nervous system. Suitable promoters include, without limitation, a retroviral RSV LTR promoter (optionally with an RSV enhancer), a CMV promoter (optionally with a CMV enhancer), a CMV immediate early promoter, an SV40 promoter, a dihydrofolate reductase (DHFR) promoter, a β-actin promoter, a phosphoglycerate kinase (PGK) promoter, an EFlα promoter, a MoMLV LTR, a CK6 promoter, a transthyretin promoter (TTR), a TK promoter, a tetracycline responsive promoter (TRE), an HBV promoter, an hAAT promoter, a LSP promoter, a chimeric liver-specific promoters (LSPs), an E2F promoter, the telomerase (hTERT) promoter, and a CMV enhancer/chicken β-actin/rabbit β-globin promoter (CAG promoter; Niwa et al., Gene (1991) 108(2):193-9). In some embodiments, the promoter comprises a human β-glucuronidase promoter or a CMV enhancer linked to a chicken β-actin (CBA) promoter. The promoter can be a constitutive, inducible, or repressible promoter.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction.

For in vivo delivery of an expression cassette for the binding member, viral transduction may be used. A variety of viral vectors known in the art may be adapted by one of skill in the art for use in the present disclosure, for example, recombinant adeno-associated viruses (rAAV), recombinant adenoviruses, recombinant retroviruses, recombinant poxviruses, recombinant lentiviruses, etc. In some embodiments, the viral vector used herein is a rAAV vector. AAV vectors are especially suitable for CNS gene delivery because they infect both dividing and non-dividing cells, exist as stable episomal structures for long term expression, and have very low immunogenicity (Hadaczek et al., Mol Ther (2010) 18:1458-61; Zaiss, et al., Gene Ther (2008) 15:808-16). Any suitable AAV serotype may be used. For example, AAV serotype 1, 2 or 9 may be used. The AAV may be engineered such that its capsid proteins have reduced immunogenicity in humans. In some embodiments, AAV1 is used because this serotype exhibits excellent parenchymal spread and while neuronal transduction predominates (like most AAV vectors), this serotype also transduces astrocytes, which may be especially amenable to high-level protein expression and secretion.

Viral vectors described herein may be produced using methods known in the art. Any suitable permissive or packaging cells may be employed to produce the viral particles. For example, mammalian or insect cells may be used as the packaging cell line.

The expression constructs such as the recombinant AAV virus may be introduced to the brain of a patient via intracranial injection, intrathecal injection, or intracisterna-magna injection.

### Applications

The expression methods of the present disclosure may be used to deliver a therapeutic binding member to the nervous system of a patient. The binding member will then be expressed and secreted from the transfected/transduced cells in the nervous system and exert its therapeutic activity locally in the nervous system such as the brain. These methods can be used to target pathogenic antigens in neurodegenerative diseases such as Alzheimer's disease (e.g., Aβ and ApoE), cerebral amyloid angiopathy, synucleopathy (e.g., α-synuclein), tauopathy (e.g., tau), or ALS (e.g., SOD-1 and TDP-43 (Pozzi et al., JCI (2019) doi:10.1172/JCI123931)), Parkinson's disease (e.g., β-synuclein), dementia (e.g., tau (Sigurdsson, J Alzheimers Dis. (2018) 66(2):855-6)), Lewy Body syndrome (e.g., α-synuclein (Games et al., J Neurosci. (2014) 34(28):9441-54)), Huntington's disease (e.g., Huntingtin (WO2016016278)), and Multiple System Atrophy (e.g., P25α and α-synuclein (Games, supra)). In a particular embodiment, the neurodegenerative disease is Alzheimer's disease. A binding member expressed locally in the nervous system will target and clear the pathogenic antigen out of the nervous system such as the brain.

Accordingly, the present disclosure provides a method of treating a neurological disease (e.g., a neurodegenerative disease) in a subject such as a human patient in need thereof, comprising introducing to the nervous system of the subject a therapeutically effective amount (e.g., an amount that allows sufficient expression of the binding member so as to cause the desired therapeutic effect) of a viral vector (e.g., an rAAV) comprising a coding sequence for the binding member for a target antigen linked operatively to transcription regulatory element(s) that are active in cells of the nervous system.

### Pharmaceutical Compositions

In some embodiments, the present disclosure provides a pharmaceutical composition comprising a viral vector such as a recombinant rAAV whose recombinant genome comprises an expression cassette for the scFv-Fc binding member. The pharmaceutical composition may further comprise a pharmaceutically acceptable carrier such as water, saline (e.g., phosphate buffered saline), dextrose, glycerol, sucrose, lactose, gelatin, dextran, albumin, or pectin. In addition, the composition may contain auxiliary substances, such as, wetting or emulsifying agents, pH buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

Delivery of rAAVs to a subject may be accomplished, for example, by intravenous administration. In certain instances, it may be desirable to deliver the rAAVs locally to the brain tissue, the spinal cord, cerebrospinal fluid (CSF), neuronal cells, glial cells, meninges, astrocytes, oligodendrocytes, interstitial spaces, and the like. In some cases, recombinant AAVs may be delivered directly to the CNS by injection into the ventricular region, as well as to the striatum and neuromuscular junction, or cerebellar lobule. AAVs may be delivered with a needle, catheter or related device, using neurosurgical techniques known in the art, such as by stereotactic injection (see, e.g., Stein et al., J Vir. (1999) 73:3424-9; Davidson et al., PNAS. (2000) 97:3428-32; Davidson et al., Nat Genet. (1993) 3:219-23; and Alisky and Davidson, Hum. Gene Ther. (2000) 11:2315-29.

Routes of administration include, without limitation, intracerebral, intrathecal, intracranial, intracerebral, intraventricular, intrathecal, intracisterna-magna, intravenous, intranasal, or intraocular administration. In some embodiments, the viral vector spread throughout the CNS tissue following direct administration into the cerebrospinal fluid (CSF), e.g., via intrathecal and/or intracerebral injection, or intracisterna-magna injection. In other embodiments, the viral vectors cross the blood-brain-barrier and achieve wide-spread distribution throughout the CNS tissue of a subject following intravenous administration. In some aspects, the viral vectors have distinct CNS tissue targeting capabilities (e.g., CNS tissue tropisms), which achieve stable and nontoxic gene transfer at high efficiencies.

By way of example, the pharmaceutical composition may be provided to the patient through intraventricular administration, e.g., into a ventricular region of the forebrain of the patient such as the right lateral ventricle, the left lateral ventricle, the third ventricle, or the fourth ventricle. The pharmaceutical composition may be provided to the patient through intracerebral administration, e.g., injection of the composition into or near the cerebrum, medulla, pons, cerebellum, intracranial cavity, meninges, dura mater, arachnoid mater, or pia mater of the brain. Intracerebral administration may include, in some cases, administration of an agent into the cerebrospinal fluid (CSF) of the subarachnoid space surrounding the brain.

In some cases, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some cases, a microinjection pump is used to deliver a composition comprising a viral vector. In some cases, the infusion rate of the composition is in a range of 1 μl/min to 100 μl/min. As will be appreciated by the skilled artisan, infusion rates will depend on a variety of factors, including, for example, species of the subject, age of the subject, weight/size of the subject, serotype of the AAV, dosage required, and intracerebral region targeted. Thus, other infusion rates may be deemed by a skilled artisan to be appropriate in certain circumstances.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. “About” can be understood as within 10%, 9%, 8%, 7%, 6%, 5%, 4%, 3%, 2%, 1%, 0.5%, 0.1%, 0.05%, or 0.01% of a stated value. Unless otherwise clear from the context, all numerical values provided herein are modified by the term “about.” It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art. In the case of conflict, the present Specification, including definitions, will control. In addition, the materials, methods, and examples are illustrative only and are not intended to be limiting.

In order that this invention may be better understood, the following examples are set forth. These examples are for purposes of illustration only and are not to be construed as limiting the scope of the invention in any manner.

### EXAMPLES

In the Working Examples below, we show that single chain antibodies (Abs) fused to an Fc domain retaining FcRn binding, but lacking Fc gamma receptor (FcγR) binding, termed a silent scFv-IgG, can be expressed and released into the CNS following gene transfer with AAV. By incorporating an Fc into a scFv-IgG design, the molecule regains the bivalency of canonical IgG providing higher avidity for multimeric targets such as aggregated amyloid, and provides the ability to modulate Fc-dependent signaling if necessary. Preserving Fc-binding to the FcRn at the brain-blood barrier may improve upon the reduction of amyloid pathology seen previously with scFv alone by enabling antibody-antigen clearance via FcRn mediated efflux from the brain. While canonical IgG expression in the brain led to signs of neurotoxicity, this modified antibody (Ab) was efficiently secreted from neuronal cells and retained target specificity. Steady state levels in the brain exceeded peak levels obtained by intravenous injection of Ab. In the transgenic ThyAPPmut mouse model of progressive amyloid plaque accumulation, AAV expression of this scFv-IgG reduced cortical and hippocampal plaque load compared to control. These findings suggest that CNS gene delivery of a silent anti-Aβ scFv-IgG is well-tolerated, durably expressed and functional in a relevant disease model, demonstrating the potential of this modality for the treatment of Alzheimer's disease and other neurological diseases.

The materials and methods used in the studies described in the following Examples are described below.

### Study Design

This study was initiated to design anti-Aβ IgGs for AAV-mediated delivery to the brain for the treatment of Alzheimer's disease. These IgG constructs were designed and initially tested in vitro 2-4 times to confirm proper expression, assembly and antigen binding activity prior to in vivo experiments. Sample sizes for C57BL/6 or SCID animal studies were set based on variability observed from previous experiments expressing transgenes in vivo using stereotaxic delivery of AAV, and are defined for each experiment. Studies testing in vivo expression were performed 2-3 times. Sample size for the ThyAPPmut mice for amyloid plaque load quantification was set to account for expected inter-animal variability in plaque formation. Based on prior studies using this line, the efficacy study was performed once n≥10 per group. Animals were randomly assigned to each group for all studies. ROI identification for automated image analysis was performed by researchers blind to the experimental conditions. All animal studies were performed according to relevant guidelines.

### AAV-IgG Designs

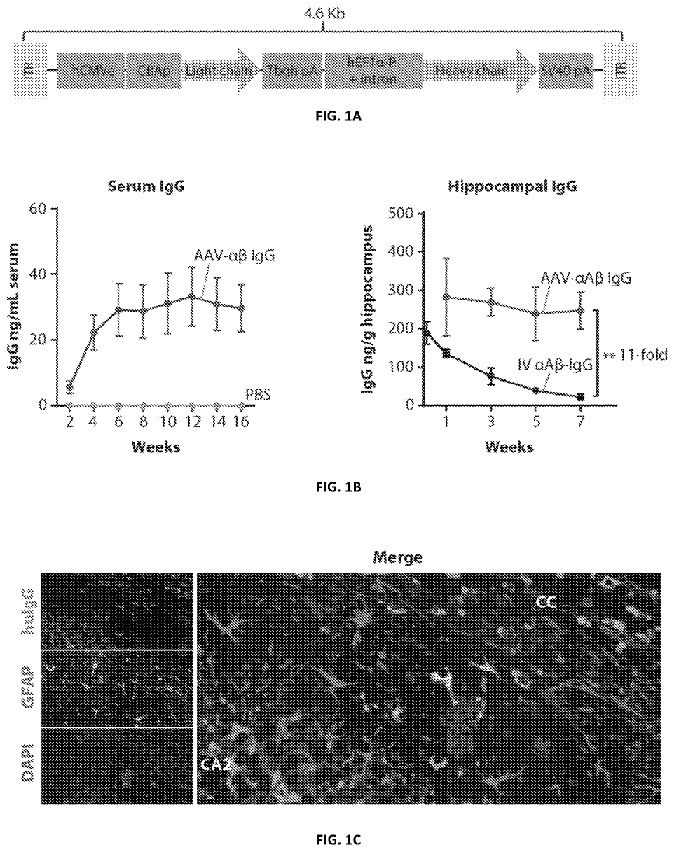

Variable regions were derived from the anti-Aβ antibody were either from the original 13C3 murine (for AAV-αAβ msIgG) or humanized sequences (for AAV-αAβ IgG) (Schupf et al., PNAS (2008) 105:14052-7), as described in patent applications WO2009/065054 and WO2010/130946, respectively. The huIgG expression vector was generated by inserting the coding sequences for the human IgG4 heavy chain containing two amino-acid substitutions described to reduce half molecules (S241P) and effector functions (L248E) (Reddy et al., J Imm. (2000) 164:1925-33) and kappa light chain into the dual promoter cassette (without the need for a 2A peptide cleavage sequence shown in FIG. 1A. For experiments requiring the mouse IgG1 framework, the original 13C3 antibody (Vandenberghe et al., Sci Rep. (2016) 6:20958) was used with the addition of an N297A mutation in the heavy chain to reduce effector function. The AAV-Control IgG vector encoded a huIgG4 PE isotype control antibody that targets a non-mammalian antigen.

### ScFv-IgG Design

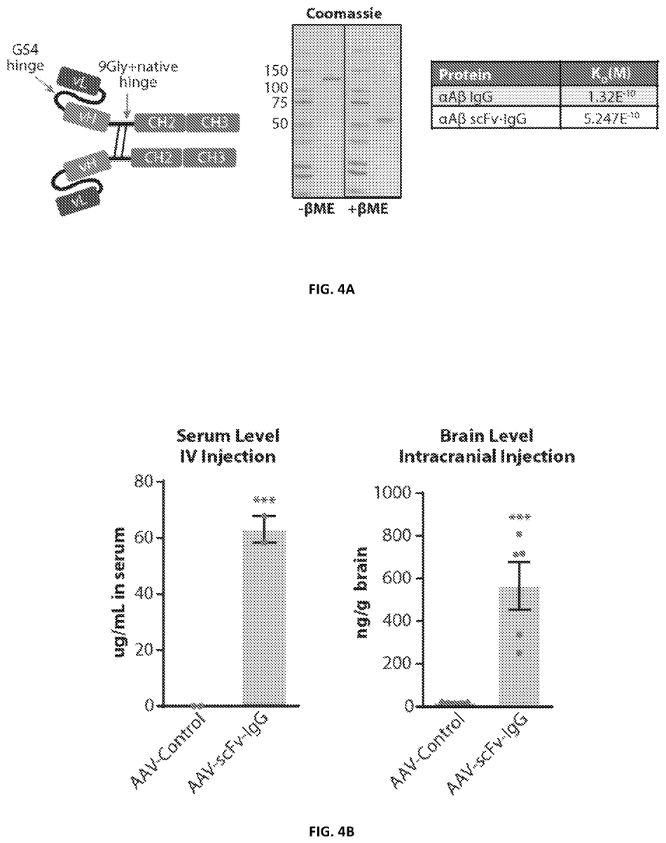

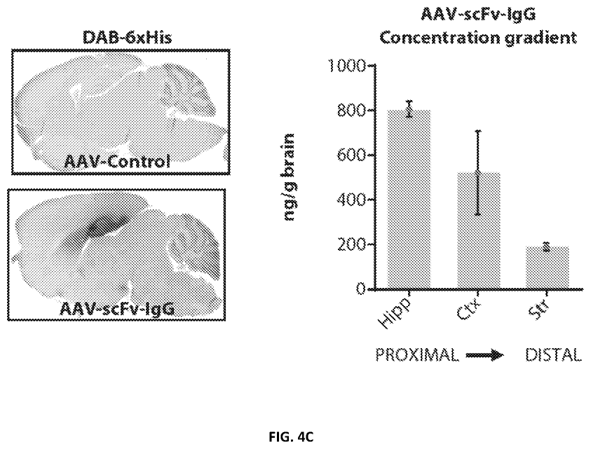

The design of the scFv-IgG is shown (FIG. 4A; SEQ ID NO: 8). Briefly, the variable light and variable heavy chain regions of the parental 13C3 anti-amyloid beta antibody were connected by 3 repeats of a flexible G₄S linker (SEQ ID NO: 2) to form a VL-VH scFv. The scFv sequence was followed by an additional 9-repeat glycine linker (SEQ ID NO: 7) (Balazs et al., Nature (2011) 481:81-4) that included the native murine IgG1 hinge and CH2 and CH3 domains to comprise the Fc region of the scFv-IgG. As with the AAV-αAβ msIgG, asparagine 297 of the Fc was mutated to alanine (N297A) to attenuate effector function (Chao et al., Immunol Invest. (2009) 38:76-92); Jefferis et al., Immunol Rev. (1998) 163:59-76). A C-terminal 6xHis epitope tag (SEQ ID NO: 9) was included to facilitate both in vitro purification and in vivo detection in mice. Expression of the scFv-IgG was driven by an hCMV/hEFla-promoter expression cassette with a Tbgh polyA.

### Immune Tolerance

To induce immune tolerance, mice were injected at days 0, 2 and 10 with 7.5 mg/kg IP with GK1.5 anti-CD4 monoclonal antibody (Bioxcell). To confirm CD4 T-cell depletion, blood was taken on day 12 by retro orbital sampling into heparin coated tubes. CD4+ T lymphocytes were quantified using FACS analysis on a BD Fortessa using standard protocols with CD45-FITC (clone 104 BD Pharmigen™), CD3e-AlexaFluor 647 (clone 17A2, eBioscience) and CD4-PE (RM4-4 clone, BioLegend) antibodies. GK1.5 treated animals had reduced CD4 as evidenced by a ratio of CD4+ lymphocytes/total CD3+ lymphocytes of 0.04+/−0.008 (mean +/−SEM) in the treated mice compared to 0.47+/−0.003 from untreated mice.

### Cell Culture, Protein Expression and Purification

Expi293™ cells (Life Tech) were passaged in Expi293T™ serum-free medium (Life Tech) and used for protein expression. The expression plasmids were transfected into Expi293™ cells via lipid transfection (Fectopro, Polyplus), and the cell culture medium containing secreted protein was collected 4 days later. Following sterile filtration, 6xHis (SEQ ID NO: 9) tagged proteins were purified via immobilized metal-affinity chromatography (IMAC). Briefly, proteins were batch adsorbed to cobalt resin (Thermo Scientific™) overnight at 4° C., washed with 10 column volumes of phosphate buffered saline, then eluted with 500 mM imidazole. Proteins were dialyzed into HEPES buffered saline overnight, concentrated (Centricon®), and frozen at −80° C. until use.

### ELISAs

96-well Immulon™ IIHB (Thermo) plates were either coated with 1 ug/mL Aβ₁₋₄₂ (Bachem H-1368) for the antigen ELISA, or 1 μg/mL mouse anti-huIgG polyclonal Ab (Jackson 209-005-088) to capture total huIgG in carbonate buffer overnight at 25° C. Wells were washed 5× in TBS-0.5% tween (TBST), and blocked in TBSTB (TBST+1.5% BSA) for 1 hr. Standard curves using purified protein were run in parallel with sera or brain homogenates to allow for quantification of bound scFv-IgG or huIgG. Samples were incubated for 2.5 hrs, washed 3× in TBST, and then incubated with HRP-conjugated secondary for 1 hr. Following 5× TBST washing, wells were incubated with TMB substrate for 5 min before quenching with 0.5 M H₂SO₄. Plate-bound signal was quantified by absorbance at 450 nm (Spectramax M5). All samples were run in triplicate.

### LC-MS/MS

The LC/MS/MS experiments were carried out on the Q Exactive™ Mass Spectrometer (Thermo Scientific™) coupled with NanoAcQuity LC system (Waters). The IgG from tissue homogenates were specifically enriched and isolated with CaptureSelect™ HuIgG affinity resins (Thermo Fisher). The enriched IgGs were digested by incubation with trypsin/Lys-C (1:100 w/w) overnight at 37° C. after DTT reduction and alkylation. The digestion was terminated by the addition of 1% formic acid (FA). The resulted tryptic peptide mixtures were loaded and separated onto a microcapillary column (75-μm id, 15 cm HSST3, 1.8 μm, Waters). Data were acquired in the PRM mode with the resolution of 70,000 (at m/z 200), AGC target 5×10⁶, and a 500 ms maximum injection time. The scheduled inclusion list was generated based on the profiling data of the control IgGs. The PRM method employed an isolation of target ions by a 2 Da isolation window, fragmented with normalized collision energy (NCE) of 25. MS/MS scans were acquired with a starting mass range of 100 m/z and acquired as a profile spectrum data type. Precursor and fragment ions were quantified using Skyline (MacCoss Lab Software).

### Surface Plasmon Resonance

Aβ₁₋₄₂ peptide (Bachem H-1368) was incubated in 10 mM HCl at 1 mg/mL overnight at 37° C., shaking at 600 rpm. The resulting fibril solution was directly immobilized on a CMS sensor chip (GE Healthcare) using amine coupling. Antibody or scFv-IgG solutions generated at 50, 30, 20, 10 and 5 nM in PBS-+P buffer (GE Healthcare) were injected at relatively high flow rate (50 μL/min) to limit avidity effects. The data were processed using Biacore™ T200 evaluation software and double referenced by subtraction of the blank surface and buffer-only injection before global fitting of the data to a 1:1 binding model.

### AAV ITR Plasmids and Adeno-Associated Viral Vector Preparation

Expression cassettes for the IgG or the scFv-IgG were subcloned into an AAV2-ITR containing plasmid, with A1AT stuffer DNA retained as needed to maintain the AAV genome size for proper packaging. In the case of the dual promoter IgG ITR plasmid, no stuffer DNA was included as the cassette was already the maximum size permitted for efficient packaging. AAV-Empty vector consisted of the CBA promoter, Tbgh polyA, and A1AT stuffer DNA. AAV2/1 virus was produced via transient transfection. In brief, HEK293 cells were transfected using PEI (polyethyleneimine) with a 1:1:1 ratio of three plasmids (containing the ITR, AAV rep/cap and Ad helper). The Ad helper plasmid (pHelper) was obtained from Stratagene/Agilent Technologies (Santa Clara, Calif.). Purification was performed using column chromatography, as previously described (Burnham et al., Hum Gene Ther Methods (2015) 26:228-42). Virus was titered using qPCR against the polyA sequence, and AAVs were stored in 180 mM sodium choride, 10 mM sodium phosphate (5 mM monobasic+5 mM dibasic), 0.001% F68, pH 7.3 at −80° C. until use.

### Animals

Animals used were C57BL/6 males obtained from Jackson Labs (Bar Harbor, USA) at 2 months of age unless otherwise specified. Adult SCID mice were obtained from Jackson Labs (B6.CB17-Prkdc^{scid}/SzJ) at 2 months of age. ThyAPPmut transgenic mice, backcrossed to C57BL/6, are described in Blanchard et al., Exp Neurol. (2003) 184:247-63. Surgical groups were housed singly to enable proper recovery from the brain surgeries. Mice were maintained on a 12-hr light/dark cycle with food and water available ad libitum. Animals were randomized to different groups and analyses were performed with operators blind to the treatment groups.

### Stereotaxic Injections

Surgery was performed according to procedures approved by the animal care and use committee. Mice were deeply anesthetized with an intraperitoneal injection of mixture (volume 10 ml/kg): ketamine (100 mg/kg; Imalgene; Merial, France) and xylazine (10 mg/kg; Rompun; Bayer, France). Before positioning the animal in the stereotaxic frame (Kopf Instruments, USA), the mouse scalp was shaved and disinfected with Vetidine (Vetoquinol, France), a local anesthetic bupivacaine (2 mg/kg at a volume of 5 ml/kg; Aguettant, France) was injected subcutaneously on the skin of the skull and Emla (Lidocaïne, Astrazeneca) was applied into the ears. During surgery, the eyes were protected from light by vitamin A Dulcis and the body temperature was kept constant at 37° C. with a heating blanket.

Samples were injected at a rate of 0.5 microliters per min. The needle was left in for 2 min to prevent flow of sample back through the needle tract, and then slowly raised out of the brain. Unilateral hippocampal injections into ThyAPPmut mice or bilateral injections into all other mice were performed. Coordinates for hippocampal injections were: AP −2.0, DV −2.0, and ML +/−1.5. Mice were kept warm and received subcutaneous injection of carprofen (5 mg/kg in a volume of 5 ml/kg, Rimadyl®, Zoetis) following surgery and observed continuously until recovery. At the end of the study, mice were euthanized by anesthetic overdose with Euthasol® (USA) or ketamine/zylazine (France). Following overdose, mice were kept warm until perfusion with ice-cold PBS.

### Immunohistochemistry

Following perfusion with cold PBS, brain tissue was fixed in 10% neutral buffered formalin (NBF). Formalin fixed tissue was embedded into paraffin, then sectioned at 5μm in the sagittal or coronal plane. All tissue was stained using a Leica BOND RX autostainer. For immunofluorescence staining, heat-mediated antigen retrieval was performed using epitope retrieval solution 1 (ER1; citrate buffer, pH 6.0) for 10 min. Tissue was then blocked/permeabilized in goat serum +0.25% triton X-100, then incubated with primary antibodies for 1 hr at RT, washed in TBST, then incubated with secondary antibodies for 30 min. Nuclei were detected using Spectral DAPI (Life). For plaque quantification tissue immunostained with biotin-conjugated 4G8 antibody (4G8 clone, BioLegend 800701) using the Vectastain® ABC (PK-7100) kit as per manufacturer's instructions without antigen retrieval or formic acid extraction.

### Antibodies

6xHis (SEQ ID NO: 9) (Abcam Ab9108, 1:1000 IHC, Invitrogen™ R931-25, 1:1000 Western, ELISA) GFAP (Ebiosciences, 41-9892-82, 1:200 or Abcam Ab4674, 1:500 IHC) 4G8 (BioLegend 800701, 1:500 IHC). Secondary antibodies from Life Technologies: Cy3 goat anti-mouse, Alexa Fluor®647 goat anti-rabbit, Alexa Fluor®488 goat anti-chicken; all at 1:500. For amyloid DAB: 4G8-biotin (BioLegend 800705 1:250).

### Image Analysis

Immunohistochemistry slides were scanned at 20× magnification using Scanscope® XT bright-field image scanner (Aperio, Vista, Calif.) or AxioScanZ1 (Carl Zeiss Microscopy GmBH, Germany). Whole slide images (WSI) of GFAP IHC were viewed and analyzed using HALO™ image analysis software (Indica Labs, Corrales, N.Mex., USA). For each WSI, the hippocampus region was manually annotated and analyzed for GFAP immunopositive area using HALO's automated area quantitation algorithm. For each sample, GFAP positive area was divided by the total tissue area for the selected ROI to obtain percent immunopositive area. For plaque analysis, 5 μm coronal brain sections were collected from 6-month-old ThyAPPmut mice at three different levels, 50 μm apart. Cortical and hippocampal ROIs were manually annotated. Amyloid plaque burden was quantified as % DAB+tissue area using a custom image analysis algorithm developed using ZEN 2 software (Carl Zeiss Microscopy GmBH, Germany). Data were plotted using GraphPad Prism version 6 (GraphPad Software, La Jolla, Calif., USA).

### Statistics

Statistical analysis was performed using Graphpad Prism (v6 and v7) using 1-way ANOVA with multiple comparisons (Dunnett) for experiments with more than two groups. Unpaired student's t test was used for comparison of two groups. *p<0.05, **p<0.01, ***p<0.001. Sample size varied, and is specified for each experiment.

### Example 1: Construction and Characterization of an AAV-IgG Vector Targeting β-Amyloid

To develop gene-based expression of an antibody, we used a dual promoter expression cassette to express a humanized version of the 13C3 antibody that binds protofibrillar and fibrillar Aβ with no affinity for monomeric forms as described in Schupf, supra. The IgG4 heavy chain included the S228P and L248E mutations that reduce Fcγ effector function and half-molecule exchange (Yang et al., Curr Opin Biotechnol. (2014) 30:225-9; Reddy et al., J Imm. (2000) 164:1925-33).

Heavy and light chains were expressed from different promoters, and the entire cassette was designed to fit within the AAV genome packaging limit (FIG. 1A). The dual promoter design used here avoids potential immunogenic or expression liabilities induced by other designs that use a single promoter, but require the use of a F2A cleavage sequence or internal ribosomal entry site for bicistronic expression (Saunders, supra; Mizuguchi et al., Mol Ther. (2000) 1:376-82). This cassette was packaged into an AAV1 capsid (AAV-αAβ IgG) for direct injection into the brain because this serotype exhibits excellent parenchymal spread and while neuronal transduction predominates (like most AAV vectors), this serotype also transduces astrocytes, which may be more amenable to high level protein expression and secretion. To test AAV-αAβ IgG expression, C57BL/6-SCID (SCID) mice were used to prevent anti-huIgG immune responses that could interfere with the expression of the transgene. Antibody is actively transported out of the brain via reverse transcytosis. Therefore, we monitored brain expression of the AAV-αAβ IgG using biweekly serum collection. Sera were drawn at 2-week intervals for 16 weeks following bilateral injection of AAV-αAβ IgG into the hippocampus (2E10 GC per side) of SCID mice. An Aβ₁₋₄₂ fibril binding immunoassay was used to measure levels of expressed, functional antibody following bilateral hippocampal injection of 2E10 GC of AAV-αAβ IgG.

The vector demonstrated stable expression for up to 16 weeks (FIG. 1B, left). To gain insight into how AAV-mediated antibody expression in the brain compares to levels observed following a standard passive immunotherapy approach, huIgG levels in the hippocampus of SCID mice were measured at different time points in parallel with a separate group that received a single intravenous (IV) bolus injection of 20 mg/kg αAβ IgG. SCID mice were injected once with 2E10 GC of AAV-αAβ IgG bilaterally into the hippocampus, or once with 20 mg/kg IV purified IgG before tissue collection at the indicated times to generate a time course of brain exposure to IgG. Ipsilateral hippocampi were homogenized and assayed for huIgG by antigen ELISA. The AAV-αAβ IgG vector sustained expression in the hippocampus of almost 300 ng/g for the duration of the time course as measured by antigen ELISA (FIG. 1B, right). Levels of IgG in the hippocampus 24 hrs after IV injection approached 200 ng/g, but these levels declined as the IgG was cleared from the brain (in line with known serum half-life), resulting in a 11-fold reduction compared to the AAV-αAβ IgG by 7 weeks.

FIG. 1C shows that intraneuronal and glial expression of AAV-IgG was detectable in the hippocampus. Specifically, expression in both neurons and astrocytes was confirmed by IHC against the huIgG expression product, with neurons readily identifiable via morphology in CA2 of the hippocampus, and colocalization with GFAP indicating astrocyte expression (FIG. 1C).

These data show that the AAV-αAβ IgG vector can maintain steady-state levels of antibody in the brain significantly higher than what can be achieved by traditional passive immunotherapy protocols.

### Example 2: Antigen Binding by AAV-αAβ IgG in a Mouse Model of Alzheimer's Disease

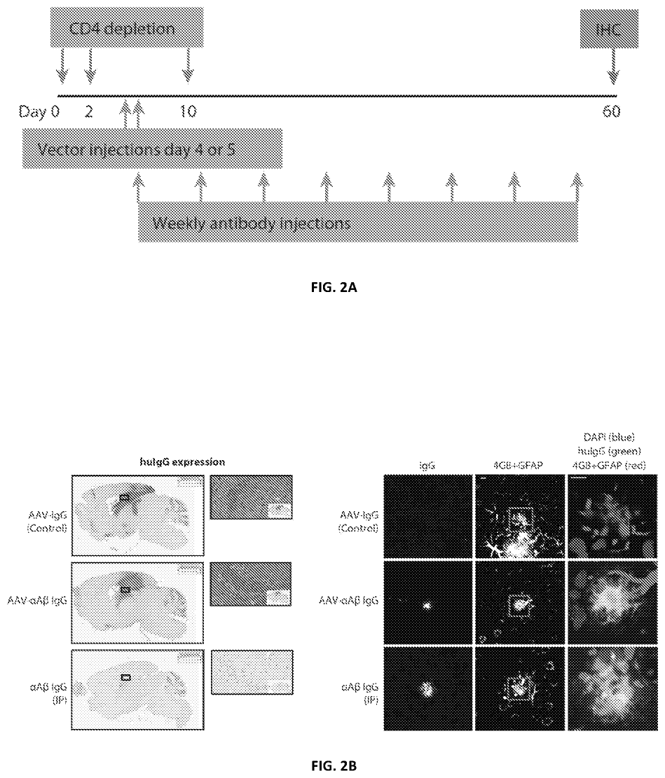

We next expressed the AAV-αAβ IgG in an amyloid plaque mouse model that expresses mutant amyloid precursor protein (ThyAPPmut) to assess the extent of brain transduction and determine whether the antibody is secreted into the extracellular space to bind plaques in vivo. This model exhibits progressive amyloid plaque accumulation in the cortex starting around 2-3 months of age (Blanchard et al., Exp Neurol. (2003) 184:247-63). To prevent anti-huIgG antibody responses, animals were immunotolerized with a CD4-depleting antibody before and after vector administration (FIG. 2A). Briefly, to readily detect the IgG in mice, we injected 2-month-old, male ThyAPPmut mice intra-hippocampally with AAV-αAβ IgG or an AAV expressing an isotype control IgG (AAV-IgG Control). ThyAPPmut mice were immunotolerized by CD4 T-cell depletion between days 2-10. AAV-αAβ IgG, or the isotype control vector AAV-IgG Control, were injected into the hippocampus bilaterally (2E10 GC per injection) at days 4-5. A separate group was injected IP weekly with purified αAβ huIgG at 10 mg/kg for the duration of the study as a positive control for plaque binding activity. After 8 weeks, 5 um sagittal brain sections were collected and immunostained. This αAβ IgG dose and IP delivery paradigm was previously shown to lead plaque binding in vivo in ThyAPPmut animals (Pradier et al., Alzheimer's & Dementia (2013) 9(4):P808-P809).

Two months after injection, at an age where these animals exhibit plaque deposition in frontal cortex, sagittal sections of brain were processed for IHC. Specifically, huIgG IHC staining revealed expression throughout the hippocampus and overlying cortex surrounding the needle track. Magnified regions of interest (ROIs) (500 μm width) show detail of huIgG expression in neurons and in the neuropil of the hippocampus. In contrast, the IP injected αAβ IgG group with staining limited to amyloid plaques did not exhibit any expression in cell bodies (FIG. 2B, left). Fluorescence IHC for huIgG, AP plaques and GFAP showed co-localization of huIgG with cortical plaques in both the AAV-αAβ IgG and the IV αAβ IgG groups, but not in the AAV-IgG control group. Specifically, AAV-αAβ IgG and peripherally delivered αAβ IgG displayed clear binding to 4G8+ amyloid deposits, while the AAV-IgG control did not display detectable binding (FIG. 2B, right).

These data show that the AAV-αAβ IgG was secreted into the extracellular space and could bind to Aβ plaques in brain regions distal to the site of injection.

### Example 3: Evaluation of AAV-αAβ IgG Neuronal Expression and Neurotoxicity

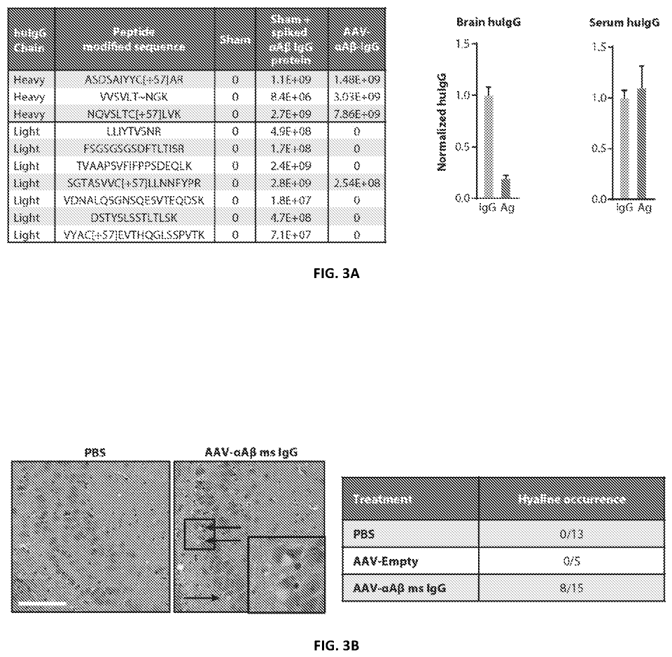

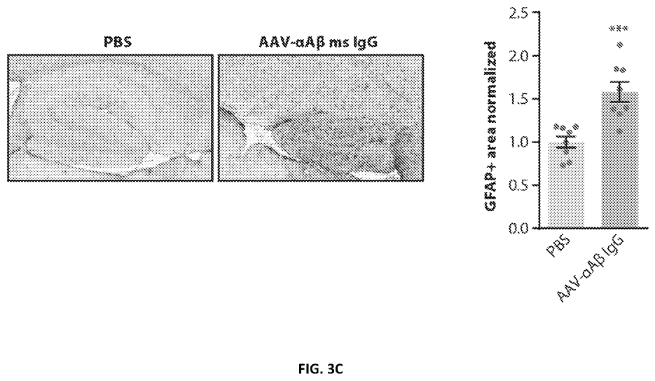

Neuronal cells are highly specialized to secrete factors relevant to neurotransmission rather than large macromolecules such as IgG. Whether efficient IgG processing and secretion can occur in these cells is unknown. To determine whether there was improper processing of the neuronally-expressed IgG, we performed mass spectrometry analysis to measure overall levels of heavy and light chains from brains after 1 month of AAV-αAβ IgG expression in SCID mice. Expression of the AAV-αAβ IgG from the hippocampus was associated with expected levels of heavy chain—similar to saline injected brain lysates spiked with purified αAβ IgG, but an unexpectedly low amount of cognate light chain when compared to the spiked control (FIG. 3A). This finding suggested that AAV-αAβ IgG expression from brain cells resulted in insufficient light chain production, resulting in an imbalance in the proportion of heavy and light chains.

We also used ELISAs to quantify total IgG (H+L chains) vs. the percent of that population that can bind antigen (Ag). Specifically, the levels of functional Aβ antibody in brain extracts from AAV-αAβ IgG expressing SCID mice were quantified by antigen ELISA, and compared in parallel with a pan-huIgG ELISA. We observed that ˜20% of the total IgG expressed from the brain (2E10 total GC injected into hippocampus) was functional, while AAV-αAβ IgG expressed from peripheral tissues via an IV injection of vector (1E12 total GC injected IV) did not have an imbalance in total IgG/functional IgG. Specifically, levels of huIgG bound to antigen accounted for only 21% of total huIgG when expressed from the brain, whereas this discrepancy was not detected in sera one month following peripheral expression of the vector (FIG. 3A, right).

We next investigated whether there was evidence for neurotoxicity as a result of IgG expression. For our initial characterizations of the AAV-αAβ IgG vector, we used the huIgG version of this antibody that has more direct translational potential for humans, and allowed for clear detection in mice. However, to test for any toxicity or neuroinflammation that could be related to brain IgG expression without the confounding variable of xenogenic huIgG exposure, we used an AAV vector termed AAV-αAβ msIgG, which expresses the original mouse version of αAβ IgG (Schupf, supra; Pradier, supra; Vandenberghe et al., Sci Rep. (2016) 6:20958). This vector was injected into the hippocampus of C57BL/6 mice and brain tissue was processed for histology one month later. Histopathological analysis revealed a high incidence of hyaline/eosinophilic cytoplasmic deposits in neuronal cells in the hippocampus, reminiscent of glycoprotein overexpression (FIG. 3B). Neuronal, eosinophilic to hyaline-like inclusions reminiscent of glycoprotein accumulation were observed only in brains injected with the antibody expression vector. These structures were also observed in the hippocampus of mice injected with the AAV-IgG Control (6/12 mice), indicating that this toxicity was not specific to αAβ IgG expression. These hyaline deposits were never observed in the hippocampus of mice injected with an AAV1-Empty vector, or PBS alone (FIG. 3B).

We also observed evidence of neuroinflammation by immunohistochemical GFAP analysis relative to PBS. In this experiment, C57BL/6 mice were injected with either PBS or AAV-αAβ msIgG (2E10 GC into hippocampus), and 5 μm sagittal brain sections were collected 16 weeks later (FIG. 3C). AAV1-Empty vector also did not elicit significant gliosis relative to PBS (1.11+/−0.12, 5 mice, mean +/−SEM normalized to PBS GFAP+ area), suggesting that the neuroinflammation was due to IgG expression.

These data indicate that while brain cells can express and secrete IgG, only a subset—about 20%—of this IgG is functional and can bind antigen, and this expression induces detectable neuroinflammation throughout the transduced region.

### Example 4: Construction and Characterization of an AAV-scFv-IgG Vector

While the IgG delivered by our vector was secreted and bound amyloid plaques in vivo, we hypothesized that an alternate Ig format could minimize the mispairing and neurotoxicity induced by the AAV-IgGs. Based on the same mouse αAβ antibody (Schupf, supra), we synthesized a modified single chain Fv, with the variable region of the IgG light chain fused to the heavy chain variable region that was connected by the COOH-terminus (C-terminus) to the murine IgG1 hinge, CH2 and CH3 domains (FIG. 4A; scFv-IgG). To minimize the pro-inflammatory effects of the Fc region, the mouse IgG1 Fc domain was mutated to eliminate glycosylation at asparagine 297 (N297A), which prevents binding to all FcγRs (Johnson, supra; Chao, supra). Specifically, the scFv-IgG was designed to have the variable regions of the murine anti-Aβ IgG linked via 3repeats of a flexible GGGGS (SEQ ID NO: 3) linker sequence. The scFv was linked to the mouse IgG1 N297A Fc via a 9-Gly repeat linker (SEQ ID NO: 7). A 6xHis tag (SEQ ID NO: 9) was added to the C-terminus. The scFv-IgG was expressed in Expi293 cells and purified by immobilized metal affinity chromatography (IMAC) using a C-terminal histidine (His) tag sequence.

Analysis by SDS-PAGE confirmed that this protein efficiently assembled into a disulfide-linked dimer (FIG. 4A). This scFv-IgG displayed binding to fibrillar Aβ₁₋₄₂ by surface plasmon resonance (SPR), comparable to the parental antibody. Affinity (M) was determined via SPR by flowing the scFv-IgG or IgG over immobilized Aβ₁₋₄₂ fibrils at different molar concentrations to analyze binding kinetics. The parental IgG exhibited an apparent dissociation constant (KD) of 1.3×10⁻¹⁰ M compared to a slightly lower binding affinity of 5.2×10⁻¹⁰ with the scFv-IgG (FIG. 4A, Table).

This expression cassette was inserted into an AAV1 vector to determine whether the modified IgG could be synthesized in vivo. IV injection of the AAV was used as a positive control for activity of our virus as peripheral tissues are well validated for expression and secretion of IgG molecules (Saunders, supra; Shimada et al., PloS ONE (2013) 8: e57606; Hicks et al., Sci Transl Med. (2012) 4:140ra187; Chen et al., Sci Rep. (2017) 7:46301; Balazs et al., Nature (2011) 481:81-4; Balazs et al., Nat Biotech. (2013) 31:647-52; Balazs et al., Nat Med. (2014) 20:296-300). One month after IV injection of AAV-scFv-IgG (1E12 total GC), serum levels reached 63 μg/mL, demonstrating robust AAV vector activity in peripheral tissues (FIG. 4B, left).

To assess brain expression of the vector, scFv-IgG levels were quantified from extracts derived from one sagittal half of the brain, termed hemibrain, one month after hippocampal injection of 2E10 total GC of AAV into C57BL6 mice. Expression levels reached a mean of ˜600 ng/g (FIG. 4B, right). Notably, this concentration was >3-fold higher than that observed 24 hrs after a 20 mg/kg IV injection of IgG, and 2.5-fold higher than that observed by AAV-αAβ IgG (FIG. 1B). Histological analysis revealed that despite having higher levels of expression in the brain than the AAV-αAβ IgG vector, AAV-scFv-IgG transduction did not cause any detectable intraneuronal hyaline protein accumulation in the injected hippocampus (0/5 mice), suggesting that the scFv-IgG was more effectively processed by neuronal cells than the IgG.

To define the brain distribution of scFv-IgG transduced cells, DAB-6xHis IHC (“6×His” disclosed as SEQ ID NO: 9) was performed on sagittal sections one month after hippocampal injection using an antibody to the His tag. The AAV-scFv-IgG vector transduced the entire hippocampus, with sparse transduction in the cortical area overlying the hippocampus around the needle track and subiculum (FIG. 4C). Brains transduced with negative control, empty AAV (AAV-Control), vectors did not show detectable anti-His immunostaining (FIG. 4C). It should be noted that anti-His IHC in C57BL6 mice only detects intracellular expression, as any secreted, extracellular scFv-IgG is likely washed away due to a lack of available antigen.

Expression of both intracellular and extracellular scFv-IgG was evaluated biochemically in ipsilateral brain regions both proximal and distal to the site of injection. One month following AAV injection, brain regions from 3 mice were dissected and expressed protein was quantified by antigen ELISA for each brain region, with PBS-injected brain homogenate used to subtract background signal. Specifically, the hippocampus, overlying cortex, and striatum were dissected and homogenized for quantification of scFv-IgG via antigen ELISA (FIG. 4C, right). A concentration gradient was observed, with highest levels detected in the injection site (hippocampus), and progressively lower levels observed in more distal brain regions (FIG. 4C, right). Despite having lower levels compared to the injection site, the concentration of the scFv-IgG in striatal tissue remained near 200 ng/g—steady state levels in the brain not typically attained by passive IgG infusion.

### Example 5: Antigen Binding by scFv-IgG in a Mouse Model of β-Amyloidosis

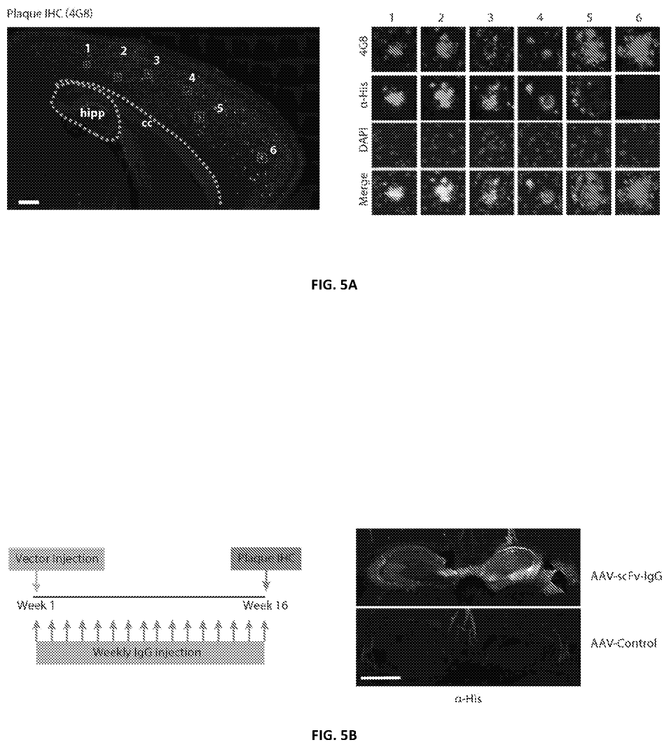

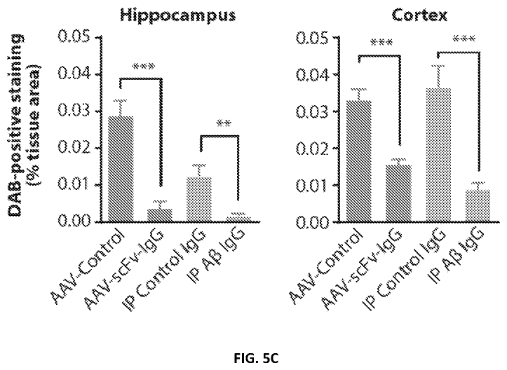

We next determined whether AAV delivered scFv-IgG was secreted into the extracellular space and could bind to antigen in vivo. The AAV-scFv-IgG vector was injected into the hippocampus of 5-month-old female ThyAPPmut mice (Blanchard, supra), an age when they have already developed plaques throughout the neocortex. 5 μm Sagittal sections of brains were processed for IHC one month after unilateral injection with 1 μL (1E10 total GC) of AAV-scFv-IgG vector and stained for His tag reactivity and Aβ plaques. Images at right show individual plaque ROIs (numbered in A) proximal (1) to distal (6) from the site of injection. Images were overlaid with 6xHis (SEQ ID NO: 9) immunostaining (green) and DAPI (blue) (FIG. 5A). As expected, abundant plaque formation was observed throughout the cortex (FIG. 5A, left) and staining with an anti-His antibody co-localized with plaques (FIG. 5A, right). Note the progressive but far reaching reduction in the intensity of 6xHis (SEQ ID NO: 9) labeling on plaques that are more distal to the hippocampus and occipital cortical areas of AAV-scFv-IgG expression, indicating that there was a clear concentration gradient of plaque-bound scFv-IgG, with plaques distal to the hippocampus showing progressively lower levels of bound scFv-IgG than plaques closer to the site of injection. These data indicate that the anti-Aβ scFv-IgG was expressed and secreted from cells in the hippocampus, which allowed it to bind to plaques distal to the injection site.

The data provided evidence that scFv-IgG delivered by the viral vector engaged its physiologically relevant target in vivo. We next determined whether long-term expression in this mouse model of amyloidosis might reduce plaque formation. Outline of study design. Four groups of 2-month-old ThyAPPmut male mice (approximate age when plaques begin to form) were injected unilaterally into the hippocampus with either the AAV-scFv-IgG, AAV-Control vector, Aβ IgG, or control isotype IgG. These groups were compared to animals treated with passive immunotherapy with weekly IP injections of the mouse anti-Aβ antibody (Aβ IgG) or an isotype control antibody at 10 mg/kg (FIG. 5B, left). Brains were collected after 16 weeks (4 months) of treatment and coronal sections were immunostained for amyloid plaques or 6×His (SEQ ID NO: 9) and analyzed for transgene expression. The AAV-scFv-IgG was expressed throughout the injected hippocampus, and there was also clear transport of vector into the contralateral subiculum, as evidenced by αHis staining of cell bodies (FIG. 5B, right). Aβ plaque load in cortex and hippocampus was quantified by anti-His IHC in coronal brain sections. ROIs from both hemispheres were combined for quantification and plaque load is expressed as DAB-positive staining as a percent of tissue ROI area. Compared to their respective controls, a single injection of the AAV-scFv-IgG caused the same magnitude of plaque reduction in the hippocampus as the αAβ IgG benchmark, despite the differences in plaque load between the control groups (FIG. 5C). Plaque reduction was also significantly reduced in cortex (FIG. 5C), consistent with evidence that the scFv-IgG diffuses from the site of expression to bind to distal plaques.

These results demonstrated that a single injection of the AAV-scFv-IgG in an amyloid mouse model was durably expressed and secreted from the site of injection to bind to plaques throughout the brain. The typical passive immunotherapy regimen of 10 mg/kg weekly anti-Aβ IgG for 16 weeks caused significant reductions in amyloid plaque formation in ThyAPPmut animals. In contrast, a single intracranial injection of the AAV-scFv-IgG resulted in comparable efficacy after 4 months of expression.

To summarize, in the above study, the scFv-IgG was derived from an antibody specific for protofibrillar and fibrillar Aβ species that reduced amyloid plaque load in vivo. Our scFv-IgG expressed well in vitro, allowing for purification and subsequent analysis of antigen binding affinity by SPR. Compared to the IgG form, the scFv-IgG bound antigen to a similar extent. AAV1 was chosen as the serotype for this indication because its capsid facilitates vector spread in the CNS following parenchymal injection. This serotype infects predominantly neuronal cells, but does transduce some non-neuronal cell types, expanding the potential repertoire of cells available for transgene expression. Using relatively high dosing (1E10 GC in the hippocampus), steady state levels at the site of injection was 3-4 times higher than what could be maximally achieved with the passive IgG benchmark we chose for comparison (antibody levels in the brain 24 hrs post-20 mg/kg IV injection of purified IgG). Dosing in the periphery of ˜60 mg/kg IV would be needed to reach the levels attained by the AAV-scFv-IgG vector.

Following a single injection, expression in the hippocampus was sustained for at least 4 months, with protein concentrations exceeding the passive immunotherapy benchmark even in brain regions several millimeters distal to the injection site. It is unlikely that transduced cells migrate from the site of injection to secrete protein in regions distal to the hippocampus, as 6xHis (SEQ ID NO: 9) positive cells were not found far beyond the site of injection or needle tract (data not shown). Long term expression of this vector in ThyAPPmut mice caused plaque reduction both in the cortex (52% reduction) and hippocampus (87% reduction). This was a more efficient reduction than that observed by other studies utilizing scFvs, where plaque reduction ranged between 0-60% (Levites, 2006, supra; Levites, 2015, supra; Kou, supra; Fukuchi, supra; and Wang et al., Brain, Behavior, and Immunity (2010) 24:1281-93). The observed magnitude of plaque reduction in animals treated with a single intracranial injection of AAV-scFv-IgG was similar to animals treated with weekly IV injections of 10 mg/kg anti-Aβ antibody for 4 months, highlighting the value of gene delivery for long term treatment paradigms.

### SEQUENCES

| SEQ ID NO | Sequence |
|---|---|
| 1 | GGGS |
| 2 | GGGGSGGGGSGGGGS |
| 3 | GGGGS |
| 4 | [GGGGS]n (n = 1, 2, 3, or 4) |
| 5 | SGGGSGGGGSGGGGS |
| 6 | GGGGSGGGGXGGGGYGGGGS (X = S, A, or N, and Y = A or N) |
| 7 | GGGGGGGGG |
| 8 | MDSKGSSQKGSRLLLLLVVSNLLLPQGVLASEIVMTQTPLSLPVSLGDRA |
| | SISCRSGQSLVHSNGNTYLHWYLQKPGQSPKLLIYTVSNRFSGVPDRFSG |
| | SGSGSDFTLTISRVEAEDLGVYFCSQNTFVPWTFGGGTKLEIKRTSSGGGG |
| | SGGGGSGGGGSEVQLQQSGPEVVKPGVSVKISCKGSGYTFTDYAMHWV |
| | KQSPGKSLEWIGVISTKYGKTNYNPSFQGQATMTVDKSSSTAYMELASL |
| | KASDSAIYYCARGDDGYSWGQGTSVTVSSASTGGGGGGGGGSGVPRDC |
| | GCKPCICTVPEVSSVFIFPPKPKDVLTITLTPKVTCVVVDISKDDPEVQFSW |
| | FVDDVEVHTAQTQPREEQFASTFRSVSELPIMHQDWLNGKEFKCRVNSA |
| | AFPAPIEKTISKTKGRPKAPQVYTIPPPKEQMAKDKVSLTCMITDFFPEDIT |
| | VEWQWNGQPAENYKNTQPIMDTDGSYFVYSKLNVQKSNWEAGNTFTC |
| | SVLHEGLHNHHTEKSLSHSPGSGSGSGSHHHHHH |

1. A method of expressing a bivalent binding member in a cell of the nervous system, comprising introducing into the cell an expression cassette encoding a polypeptide comprising an antibody heavy chain variable domain (V_{H}), an antibody light chain variable domain (V_{L}), and an IgG Fc region, wherein the V_{H} and the V_{L} form an antigen-binding site that binds specifically to a target protein, and upon expression in the cell, two molecules of the polypeptide form a disulfide-bonded homodimeric bivalent binding member specific for the target protein.

2. The method of claim 1, wherein the cell of the nervous system is a neuron; a glial cell, optionally selected from an oligodendrocyte, an astrocyte, a pericyte, a Schwann cell, and a microglia cell; ependymal cells; and brain epithelial cells.

3. The method of claim 2, wherein the cell is a human cell.

4. The method of claim 3, wherein the cell is in the brain of a patient.

5. The method of claim 1, wherein the target protein is a protein expressed in the brain.

6. The method of claim 5, wherein the protein is amyloid beta peptide (Aβ), tau, SOD-1, TDP-43, ApoE, or α-synuclein.

7. The method of claim 1, wherein the polypeptide comprises, from N terminus to C terminus,
(i) the V_{H}, a peptide linker, and the V_{L}; or the V_{L}, a peptide linker, and the V_{H}; and
(ii) the IgG Fc region.

8. The method of claim 7, wherein the peptide linker comprises the sequence GGGGS (SEQ ID NO: 3).

9. The method of claim 1, wherein the bivalent binding member binds to neonatal Fc receptor (FcRn), but it does not bind to an Fc gamma receptor due to one or more mutations in the IgG Fc region.

10. The method of claim 1, wherein the introducing step comprises administering a recombinant virus whose genome contains the expression cassette.

11. The method of claim 10, wherein the recombinant virus is introduced to the brain of a patient via intracranial injection, intrathecal injection, or intracisterna-magna injection.

12. The method of claim 10, wherein the recombinant virus is a recombinant adeno-associated virus (AAV).

13. The method of claim 12, wherein the recombinant AAV is of serotype 1 or 2.

14. The method of claim 1, wherein expression of the polypeptide is under the transcriptional control of a constitutively active promoter or an inducible promoter.

15. The method of claim 4, wherein the patient has a neurodegenerative disease.

16. The method of claim 15, wherein the patient has Alzheimer's disease, cerebral amyloid angiopathy, synucleopathy, tauopathy, or amyotrophic lateral sclerosis.

17-18. (canceled)


SEQUENCE LISTING (9 sequences)

SEQ ID NO: 1 - protein, 4 aa, Artificial Sequence
  source: /note=“Description of Artificial Sequence Synthetic peptide”
GGGS

SEQ ID NO: 2 - protein, 15 aa, Artificial Sequence
  source: /note=“Description of Artificial Sequence Synthetic peptide”
GGGGSGGGGSGGGGS

SEQ ID NO: 3 - protein, 5 aa, Artificial Sequence
  source: /note=“Description of Artificial Sequence Synthetic peptide”
GGGGS

SEQ ID NO: 4 - protein, 20 aa, Artificial Sequence
  source: /note=“Description of Artificial Sequence Synthetic peptide”
  SITE (1)..(20): /note=“This sequence may encompass 1-4 ‘Gly Gly Gly Gly Ser’ repeating units”
GGGGSGGGGSGGGGSGGGGS

SEQ ID NO: 5 - protein, 15 aa, Artificial Sequence
  source: /note=“Description of Artificial Sequence Synthetic peptide”
SGGGSGGGGSGGGGS

SEQ ID NO: 6 - protein, 20 aa, Artificial Sequence
  source: /note=“Description of Artificial Sequence Synthetic peptide”
  VARIANT (10)..(10): /replace=“Ala” or “Asn”
  VARIANT (15)..(15): /replace=“Asn”
  SITE (1)..(20): /note=“Variant residues given in the sequence have no preference with respect to those in the annotations for variant positions”
GGGGSGGGGSGGGGAGGGGS

SEQ ID NO: 7 - protein, 9 aa, Artificial Sequence
  source: /note=“Description of Artificial Sequence Synthetic peptide”
GGGGGGGGG

SEQ ID NO: 8 - protein, 530 aa, Artificial Sequence
  source: /note=“Description of Artificial Sequence Synthetic polypeptide”
MDSKGSSQKGSRLLLLLVVSNLLLPQGVLASEIVMTQTPLSLPVSLGDRASISCRSGQSLVHSNGNTYLHWYLQKPGQSPKLLIYTVSNRFSGVPDRFSGSGSGSDFTLTISRVEAEDLGVYFCSQNTFVPWTFGGGTKLEIKRTSSGGGGSGGGGSGGGGSEVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTGGGGGGGGGSGVPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGSGSGSGSHHHHHH

SEQ ID NO: 9 - protein, 6 aa, Artificial Sequence
  source: /note=“Description of Artificial Sequence Synthetic 6xHis tag”
HHHHHH